(19) United States
(12) Patent Application Publication
(10) Pub. No.: US 2013/0331601 A1
(43) Pub. Date: Dec. 12, 2013

(54) METHOD FOR RECOVERY OF ORGANIC ACID FROM DILUTE AQUEOUS SOLUTION

(71) Applicants: Esko Tirronen, Espoo (FI); Jukka Hietala, Porvoo (FI); Reijo Aksela, Espoo (FI)

(72) Inventors: Esko Tirronen, Espoo (FI); Jukka Hietala, Porvoo (FI); Reijo Aksela, Espoo (FI)

(21) Appl. No.: 13/992,343

(22) PCT Filed: Dec. 9, 2011
(86) PCT No.: PCT/FI11/51096

§ 371 Date: Aug. 14, 2013

(30) Foreign Application Priority Data
Dec. 10, 2010 (FI) 20106311

(51) Int. Cl. C07C 51/493

(52) U.S. Cl. CPC C07C 51/493
USPC 560/248; 562/609; 562/608

(57) ABSTRACT

The present invention relates to a method and an arrangement for recovery of at least one organic acid from a dilute aqueous solution thereof. In this method a complex between said organic acid and an extractant is formed by contacting the solution with a reactive extractant and dissolving the formed complex into said extractant thus forming an extractant phase. The organic acid is removed from the formed complex by esterification using an alcohol.

### FIELD OF THE INVENTION

The present invention relates to a method for isolating and recovering organic acid or acids from aqueous solutions thereof wherein the amount of organic acid is low. In particular, the method relates to isolation of pure carboxylic acids from their dilute aqueous solutions.

### BACKGROUND OF THE INVENTION

Several industrial scale processes are known to produce dilute aqueous waste solutions comprising low amounts of carboxylic acids. Due to environmental reasons recovery of these acid components has become increasingly important. These economically valuable by-products have typically been recovered by distillation or extractive distillation which are effective but energy consuming and technically challenging processes due to formation of azeotropes or stable emulsions rendering processing uneconomical or providing the product in an undesirable form, such as too dilute solution, which is difficult to use in further processes.

In many cases, the carboxylic acids generated as the result of biomass degradation are obtained as dilute aqueous solutions. Distillation is an obvious method to purify isolated substances from aqueous solutions, but distillation as such is not the best option as far as energy-efficiency is considered. Besides, some of the components such as formic acid may form azeotropes with water making the separation into pure components difficult. The separation can be accomplished by arranging several distillation processes and equipment parallel or in series but then the energy cost of separation and equipment will become high. Furthermore, separation into single components is not feasible without using large distillation columns with a high number of separation stages or trays.

Separation of various chemicals may be based on liquid-liquid extraction processes. Even carboxylic acids have been separated from dilute aqueous solutions with extraction solvents insoluble or slightly soluble in water, or with solvent mixtures. However, the efficiency of extraction agents is typically not satisfactory enough to yield pure components.

DE 19747789 discloses extracting carboxylic acids from dilute aqueous solutions with an extractant mixture comprising trialkyl amine containing at least 15 carbon atoms and secondary amide containing more than 7 carbon atoms which is liquid at 25° C. Similarly, DE 19747791 discloses extracting carboxylic acids with an extractant mixture comprising the trialkyl amine and alcohol containing 3-5 carbon atoms. Both methods use considerable amount of organic extractant solution compared to the aqueous acid to be recovered, such as more than 1:1. Subsequently, carboxylic acids are separated from the extractant mixture by rectification.

Organic acids can be effectively extracted from dilute water solution using reactive extractants such as trialkyl phosphine oxides as disclosed in U.S. Pat. No. 3,816,524. In this method dilute aqueous solution containing lower C1-C4 mono or dicarboxylic acids is contacted with a liquid water-immiscible organic solvent comprising one or more trialkyl phosphine oxides. The carboxylic acid is efficiently extracted into the extractant. It is disclosed that any appropriate method may be used to remove the extracted acid from the loaded extractant. Specifically named methods include stripping by water or formation of an alkali solution. Alternatively, the extracted acid may be converted into ammonium carboxylate.

It has been found that stripping with water, even with hot water is not an applicable method, especially the yield of the acid to be recovered remains low. Furthermore, the acid is recovered in a very dilute form due to the need for extensive use of water in stripping. Recirculating large amounts of liquids is energetically unfavourable and requires massive processing equipment and frequent maintenance.

Stripping with an alkaline solution produces a carboxylic acid salt the conversion of which back to an acid and hydroxide is complicated and uneconomical. Carboxylic acid can be liberated from its salt form by treatment with a stronger acid, like sulphuric acid. The side product is an inorganic salt with low value. For example, if lye is used in stripping and sulphuric acid in recovery of formic acid:

HCOOH+NaOH⇄HCOONa+H₂O

2HCOONa+H₂SO₄→2HCOOH+Na₂SO₄

Sodium sulphate produced cannot be reacted back to NaOH economically and it must be considered as waste in the process.

It is also possible to convert a carboxylic acid salt to a free acid by treatment with an acidic ion exchange resin but the resin needs to be regenerated with a stronger acid, which produces the same inorganic waste salt as in the previous case.

Typically extractants will contain low amounts of extracted acids which leads to high amount of required extractants. Treatments with high amounts of organic solvents are in general problematic especially in industrial scale. Therefore, many methods aim at decreasing the amount of solvents to be circulated even at the expense of introducing complex processing configurations. WO0127063 discloses contacting carboxylic acid containing aqueous solution with a water-insoluble amine solvent. The acid is extracted from the aqueous phase into the organic phase and forms an extract carrying amine-bound carboxylic acid. The extract is split into two streams, the first stream which is back-extracted with water and concentrated to form a concentrated solution for recombination with said second extract stream. The extract is loaded using the concentrated solution and the loaded extract is reacted to form a non-ionized derivative, such an ester. When ester is formed, the amine solvent is liberated for recycling.

Very effective derivatives based on trialkylphosphine oxides have been developed and are commercially available by the name of CYANEX®. An extractant especially effective for for example acetic acid extraction is CYANEX® 923 comprising a mixture of four trialkylphosphine oxides which effectively extracts the acid from an aqueous solution and forms a stable complex.

GB2191490 discloses an extraction process for the recovery of organic acids such as citric, malic, tartaric or oxalic acid from aqueous solutions using a mixture of tri-alkyl phosphine oxides having a cyanex-type formula of (R₁,R₂,R₃)P(O) wherein each R₁, R₂ and R₃ is the same or different alkyl group of 2 to 10 carbon atoms, the total number being from 15 to 27. The aqueous solution of the organic acid is contacted with extractant mixture and an organic solvent comprising an aliphatic hydrocarbon, aromatic hydrocarbon kerosene, sulfonated kerosene or ether. The extracted organic acid is subsequently re-extracted from the extractant using distilled water.

One problem in the recovery of organic acids by extraction from dilute solutions thereof, such as fermentation solutions, is the formation of stable emulsions due to proteins and other unknown substances existing in the solution to be treated.

Another problem is that in reactive extraction efficient recovery of the extracted acids, such as carboxylic acid, from the extractant is challenging. For example, trialkylphosphine oxide type extractants form very stable complexes with carboxylic acids which are difficult to break by conventially used thermal treatment or by back extraction even with hot water. The yield of the recovered acid remains low, or the separation is energy consuming and economically non-feasible, or the concentration of recovered organic acid remains very low.

A further problem is separating mixtures of organic acids from dilute aqueous solutions thereof. The recovery of extracted acids from the extractant is difficult in acid form due to small differences in boiling points or possible azeotrope formation tendencies if recovered back to dilute aqueous solutions.

Preparation of esters directly from dilute aqueous solutions containing water soluble organic acids is known. For example in GB933714 it is described how glacial acetic acid was dissolved in water and then mixed with methanol, sulphuric acid and xylene. The mixture was heated while being thoroughly mixed and then the organic phase was separated off. By distilling it, 74% of the originally added acetic acid was obtained in the form of methyl acetate. WO2005070867 discloses a reactive extraction method for the recovery of levulinic acid from an aqueous mixture containing e.g. levulinic acid, formic acid and furfural wherein the mixture is first contacted with a liquid esterifying water-immiscible alcohol, such as 1-pentanol, in the presence of a catalyst at 50 to 250° C. to form esters of levulinic acid, such as pentyl levulinate, and formic acid, such as pentyl formate. These esters remain in organic phase together with the alcohol and furfural. The desired levulinate and also the other compounds can be separated by applying different sequential separation methods, distillations such as e.g. reactive distillation from the organic phase. Formic acid ester is converted to formic acid by acid hydrolysis and separated simultaneously by distillation from the alcohol. Formic acid is equally obtainable as an ester from the organic phase requiring further processing for the recovery of the pure acid.

The object of the present invention is to provide an efficient method for recovery of carboxylic acid(s) from dilute aqueous solutions.

A further object of the present invention is to obtain carboxylic acid(s) as concentrated pure compound(s) with good yield.

Yet, a further object of the present invention is to recover carboxylic acid(s) efficiently without using extensive amounts of organic solvents in the recovery process.

### SUMMARY OF THE INVENTION

The present invention is directed to solve the problems presented. The inventors have found that combining an organic acid extraction process with subsequent esterification, and optionally hydrolysation, high purity concentrated carboxylic acid is obtained from dilute aqueous solution thereof. The overall process is economical and efficient as the acid separation is facilitated and the process provides high acid yields and the amount of water circulating within the process or to be removed from the process is low.

Furthermore, separation and recovery of multiple carboxylic acids from a mixture thereof in a dilute aqueous solution is facilitated.

The present invention provides a method for recovery of at least one organic acid from a dilute aqueous solution thereof as depicted by claim 1. An arrangement suitable for use in said method is depicted by claim 16.

However, as far as known to the present inventors, esterification has not been used as a technique to efficiently liberate an acid from a strongly bonding active extractant. Use of strongly bonding active extractant has the advantage that it removes the acids efficiently from dilute aqueous solutions and esterification takes then place in essentially water free environment. Esterifications are equilibrium reactions where high water concentration favours ester hydrolysis back to free acid and alcohol.

The advantages of the process according to the present invention are especially the high carboxylic acid yields mainly due to the use of strongly bonding active extractant and the possibility to recover the acids in concentrated form due to subsequent esterification in combination with the used extractant. There is thus a synergistic effect in coupling the strongly bonding extracting step with the subsequent stripping of acid by an esterification step. Moreover, the separation of multiple acids in ester form is more convenient than separation in acid form. The acids' technically challenging tendency to forming azeotropes and the subsequent purification steps and recovery of the acids therefrom can be avoided.

In the process of the present invention recirculation of chemicals used therein such as alcohol and extractant is possible resulting in economical and versatile processing of pure pure acids or acid esters depending on the desired end product or application. One factor favouring the economics is the possibility to avoid high energy consuming evaporation of considerable amounts of water. The amount of chemicals to be used and recycled remains reasonable due to a diminished organics to acid ratio producing secondary advantages in terms of more compact processing apparatus requiring less energy and facilitating the maintenance. Moreover, methyl esters of carboxylic acids have typically lower boiling points than the free acids which favors separation by distillation in ester form.

### BRIEF DESCRIPTION OF THE DRAWINGS

FIG. 1 is a schematic layout set up for an arrangement suitable for use according to the method of the present invention combining extraction, esterification and optional hydrolysation.

FIG. 2 is a schematic flowchart of one possible apparatus and process for production of concentrated organic acid, such as formic acid, according to the present invention.

FIG. 3 is a schematic flowchart of one possible apparatus and process for production of multiple organic acid esters, such as formic acid and levulinic acid esters, according to the present invention.

### DETAILED DESCRIPTION OF THE INVENTION

Many industrial scale processes produce as a waste product dilute aqueous solutions containing low concentrations of organic acids, such as carboxylic acids. These dilute aqueous solutions containing organic acids may originate from a variety of different industrial processes and sources producing biomass such as from pulp industry, waste paper handling, paper mill sludge, urban waste paper, agricultural residues, rice straw, woody plant, cotton materials and cellulose fines from papermaking or any biomaterial processing such as fermentation.

The dilute aqueous solution of the present invention preferably originates from industrially used biomass such as biomass from petrochemical plants or wood pulping mills, more preferably from processing such biomass, most preferably from processes were biomass is treated thermally, chemically or biologically to produce useful reaction products. Preferably, carboxylic acids therein are the desired products or byproducts of the original process. The aqueous solution of the present invention can be as such a waste stream. The biomass preferably contains acid sources such as sugars and their oligomers and their polymers like cellulose and starch. Treatment of biomass is usually performed in presence of a large amount of water. Therefore, the concentration of organic acids in the end product is usually low, such as less than 15 wt-%, especially if the acid is removed as a condensate from the main process stream.

The amount of organic acid, preferably carboxylic acid, in the dilute aqueous solution of the present invention is below 40% by weight. Preferably, the concentration of the organic acid in the dilute aqueous solution to be processed is less than 15% by weight, more preferably from 0.01 to 10%, most preferably from 0.1 to 7%, such as from 0.5 to 5%. This is the total amount of organic acids such as carboxylic acids, to be recovered in case there are several acids to be recovered simultanously.

In one aspect of the present invention a method is provided wherein at least one organic acid is recovered from a dilute aqueous solution thereof comprising the steps of extraction, esterification, and optionally hydrolysis.

In the extraction step, a complex between the organic acid with the extractant is formed by contacting the dilute aqueous solution comprising the organic acid component with a reactive extractant. A complex between the organic acid and the extractant is formed which is soluble into the extractant, preferably liquid extractant, and forms an extractant phase. The extractant may comprise hydrocarbon diluents for adjustment purposes such as viscosity adjustment, but preferably the active extractant is used as such, pure, in order to minimize the amount of organic solvent to be incorporated into the processing.

Before the esterification step, the aqueous solution phase and the extractant phase are separated from each other. The aqueous solution phase which is depleted from the desired organic acid(s) is removed from said extractant phase which is processed further.

In the subsequent esterification step, the organic acid(s) is (are) removed from the formed complex in the extractant phase by esterification using an alcohol. The corresponding organic acid ester(s) is(are) formed. The esters are optionally separated from the extractant phase.

Optionally, the formed and separated ester(s) is (are) subsequently hydrolysed into corresponding acid(s) and alcohol. The obtained acid(s) is(are) collected.

The organic acid in the dilute aqueous solution of the present invention comprises at least one carbon containing acid that is at least to some extent soluble in water. The organic acid is preferably a carboxylic acid or mixtures threreof, more preferably C1-C10 carboxylic acid, most preferably aliphatic C1-C5 carboxylic acid, such as formic acid, acetic acid, propionic acid or levulinic acid or mixtures thereof.

Some of the carboxylic acids, especially formic acid and propionic acid, form azeotropes in aqueous solutions which renders the separation of pure acids difficult or even impossible by distillation from dilute solutions thereof.

Several separation methods have been conventionally employed to recover carboxylic acids from fermentation liquors. These processes comprise ultra filtration, reverse osmosis, electro-dialysis, distillation, anion exchange, precipitation, adsorption and liquid extraction. Reactive extraction was found to be especially useful in separating efficiently the carboxylic acid from the aqueous solution. The outcome of the reactive extraction strongly depends on the selection of a suitable extractant, degree of extraction, the loading ratio, complexation equilibrium constant, type of complex formed, rate constants, properties of the extractant solvent, temperature, pressure, pH and acid concentration.

The reactive extractant according to the method of the present invention is selected from extractants having as high partition coefficient as possible. Preferably the extractant contains at least one of the groups RR′R″P═O, RR′R″N, H(C═O)NRR′ or R(C′O)NR′R″ as the complex forming group wherein R, R′ and R″ are the same or different C1-C20 carbon chains. More preferably, the extractant is selected from the group of trialkyl phosphine oxides, N,N-dialkyl amides, trialkyl amines and dialkylformamides, preferably in liquid form. Most preferably, the extractant is selected from trihexylphosphine oxide, dihexylmonooctylphosphine oxide, dioctylmonohexylphosphine oxide, tri-n-octylamine, tri-n-(octyl-decyl)-amine, tris(isooctyl)amine, N,N-dibutylformamide and mixtures thereof. The extractant as such or dissolved in a diluent or a mixture of diluents is capable of forming complexes with organic acid(s) which increases the concentration of organic acid(s) in the extractant phase. Preferably, the extractant is used as such i.e. without the need to dissolve it into any additional diluent whereby the ratio of extractant to aqueous solution may be minimized.

The extraction may be performed and parameters and conditions chosen as commonly known form several earlier publications.

The volume ratio of the dilute aqueous solution containing the organic acid component to the extractant during extraction depends on the details of the selected process but is such that at least 50%, preferably at least 80% and most preferably at least 95% of the desired acid can be removed from the aqueous solution to the extractant.

In general, due to the used extracants the volume ratio of organics to aqueous or acid phase is large, such as over 1. In the present invention this ratio is less than 0.75, preferably less than 0.5, more preferably less than 0.3 which leads to high efficiency and economical result, especially in industrial scale operation.

The extraction may be performed in batch or continuous mode. Preferably, a counter current liquid-liquid extraction column is used, operating in continuous mode.

The extractant phase comprises the extracted organic acid from the dilute aqueous solution, the extractant, optionally possible diluents and the formed complex which is dissolved into the extractant. In addition, there may be present some water residue, preferably less than 5% by weight.

The formed strong organic acid-extractant complex is difficult to break with conventional processes such as thermal treatment or by back extraction with, for example, hot water. High temperatures during thermal treatment increase the risk to thermal decomposition of the acids, especially in the case of formic acid. Back extraction with water leads to yield losses or formation of dilute acids and azeotropes due to need for extensive use of water.

In the method of the present invention the acid is released from the organic acid-extractant complex by forming an ester thereof.

Alcohol is added to the separated organic extractant phase. The temperature of the alcohol-organic extractant phase solution is elevated and the esterification reaction is preferably performed at ambient pressure. The organic acid is removed from the complex by formation of the corresponding ester which is preferably recovered by, for example, distillation.

Especially, if multiple organic acids are to be recovered simultaneously it is advantageous to separate them in ester form by fractionating distillation. The separation of multiple organic acids in the form of esters is easier due to more enhanced boiling point separation compared to separation of the corresponding acid forms.

The alcohol used for esterification comprises C1-C6 alcohols, preferably methanol or ethanol, which give the lowest boiling esters. The alcohol is preferably selected in a way that the ester has as low boiling point as possible but does not distil together with any component of the mixture. For example, fermentation broths can contain small amounts of complex mixtures of various volatile components that are extracted together with the organic acids. The method of the present invention makes it possible to distil the acids as esters at temperatures where these impurities do not have any effect to the product purity.

The elevated temperature in esterification is selected based on the extracted acid, alcohol and extractant used. Temperature is preferably selected in a way that it drives both esterification and distillation of the ester at same time. The esterification is preferably continued as long as distillate is produced or depending on the desired yield and process time to a certain optimized value to be determined by the man skilled in the art.

Alcohol is used in molar excess to acid, preferably in excess of more than 0.1, more preferably in excess of 0.5-4, most preferably in excess of 0.8-3, to drive the esterification. In case of a mixture of acids, the amount of alcohol can be selected in a way that the kinetically favoured ester can be primarily recovered from the mixture.

In one embodiment an acid catalyst is used to enhance the esterification. For example, formic acid as such has a catalytic effect for the esterification reaction. Any conventional esterification catalyst can be used, preferably p-toluenesulphonic acid, mineral acid such as sulphuric acid, or acidic ion exchange resin. Solid catalysts may be used as structured elements inside the reactor or reaction column.

The esterification reaction is an equilibrium reaction. In a continuous process the constant withdrawal or removal of the formed ester(s) or formed water shifts the reaction equilibrium resulting in further formation of the ester which is typically preferred. A major advantage of the present process is the low amount of residual water to be circulated within the process. The possibility to recycle the used and recoved extractant and esterification chemicals further favours the efficiency and ecomonics of the process.

In a preferred embodiment the possible excess alcohol used in esterification and alcohol released in hydrolysis of the ester are preferably recycled back to esterification.

In another preferred embodiment the resulting free extractant is reused and preferably recycled back to the extraction step as such or after a purification step.

In one embodiment the extractant is counter currently contacted with the alcohol using an ion exchange resin column wherein the esterification reaction takes place.

The ester produced may be the end product as such, or it is optionally processed further into the corresponding carboxylic acid. The optional process preferably includes hydrolysis of the obtained carboxylic acid ester. There are presently available several ways to obtain pure organic acid, such as formic acid from methyl formate, using hydrolysis. Preferably the hydrolysis is performed according to the method of EP0005998. Pure formic acid may be produced in a continuous process by hydrolyzing methyl formate with water at elevated temperature and pressure, preferably in the presence of a formic acid catalyst. The formed methanol is separated from the formic acid and preferably recycled back to the esterification step. It is possible to obtain very high purity concentrated formic acid, preferably over 20% by weight directly from hydrolysis unit enabling economical rectification, or more preferably about 35%, most preferably about 85%, such as even 99% by weight, from methyl formate hydrolysis using the method described in EP0005998.

In one embodiment of the present invention methyl formate is fed through an ion exchange resin column, preferably an ion exchange bed, in which the hydrolysis into formic acid and methanol, and the separation of formic acid from methanol take place simultaneously by means of the catalytic and adsorbent properties of a solid in exchange material in the ion exchange as described in the applicant's previous patent application U.S. Pat. No. 6,429,333 wherein e.g. a conversion of 0.78 and a formic acid concentration of 22% by weight were achieved at room temperature, under atmospheric pressure, and with a water/methyl formate ratio of 1:2 as depicted by FIG. 4 of the application.

The alcohol obtained from the hydrolysis is preferably reused and recycled to the infeed of the esterificaltion step. If considered necessary the alcohol may be purified by known means before infeed. Preferably the alcohol which is recycled back to esterification has a water content less than 10%.

In another aspect of the present invention an arrangement suitable for carrying out the above described method is provided. This arrangement comprises an extraction unit for carrying out an extraction of at least one organic acid from dilute aqueous solution thereof with an extractant forming a complex between said organic acid and said extractant. The extraction unit is connected to at least one esterification unit for carrying out esterification of said extracted acid from said complex. Optionally the esterification unit is connected to a hydrolysis unit for carrying out hydrolysis of said esterified organic acid into free organic acid and alcohol.

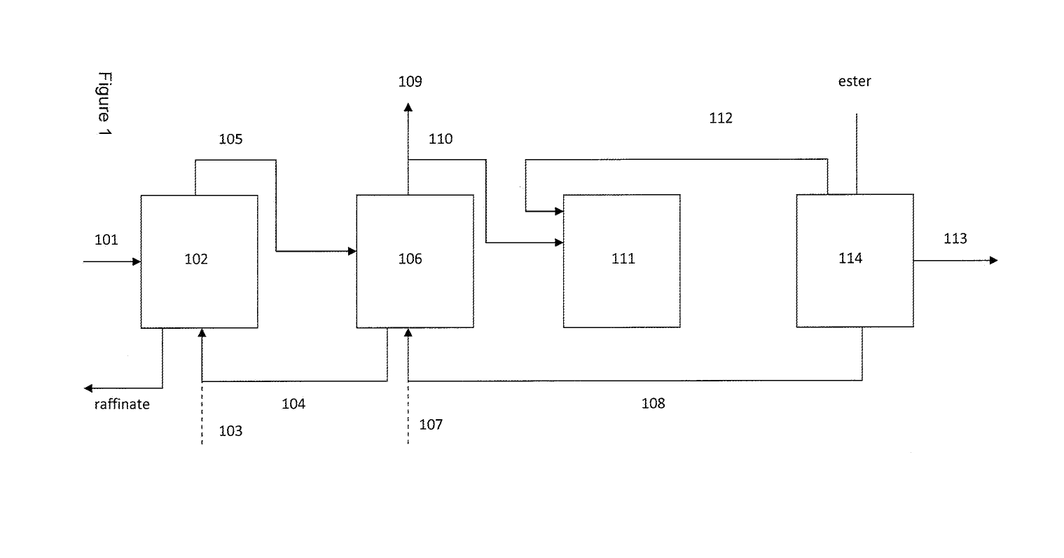

FIG. 1 illustrates one possible schematic layout for a suitable set-up combining extraction, esterification and optional hydrolysation.

Based on the layout of FIG. 1, dilute aqueous acid containing solution 101 is fed into an extraction unit 102 together with fresh 103 or recycled 104 extractant. The formed liquid extract 105 comprising essentially extractant and the complex formed between the extractant and acid and some residual aqueous infeed is fed into an esterification unit 106 together with fresh 107 or recycled 108 alcohol. The esterification unit optionally comprises several esterification unit in case of multiple acid to be recovered. Preferably these units are in series wherefrom separate acid and alcohol streams are directed individually into equivalent optional hydrolysis units. The formed ester(s) may be used as such 109 or is(are) processed further 110. Optionally, an ester is directed to a hydrolysis unit 111 together with water 112 and hydrolysed back to respective acid 113 and alcohol 108 which is recycled back to an esterification unit 106. Depending on the need the hydrolysis product(s) is(are) processed further 114 using separations or preferably distillations for recovery of acid(s) in concentrated form.

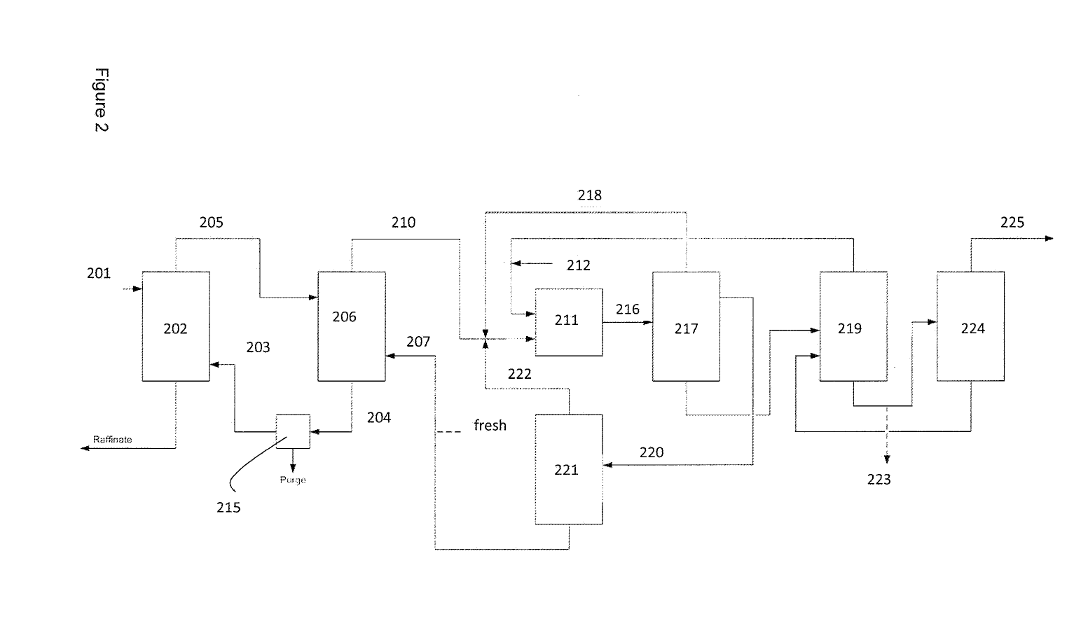

In a preferred embodiment of FIG. 2 concentrated organic acid such as formic acid, is produced from a dilute aqueous solution thereof. The dilute aqueous acid solution 201 is fed counter currently into an extraction unit 202 together with extractant 203. The organic extract phase 205 containing the formed complex of acid and extractant is directed into an esterification unit 206 together with alcohol 207 such as methanol. The formed ester 210, such as methyl formate, is removed from the esterification unit after distillation and directed into hydrolysis unit 211 together with water 212. The recovered extractant 204 is directed into a purification unit 215 and recycled back to extraction unit 206 together with fresh extractant. After hydrolysis the formed acid-water mixture 216 is directed into separation unit 217 wherefrom the residual ester phase 218 is recirculated back to hydrolysis infeed and the acid-water mixture is directed into water distillation 219. The separated alcohol-ester residue mixture 220 is directed into further separation for recycling the alcohol component 207 back to esterification unit and ester residue 222 to hydrolysis. Remaining water 212 is distilled from the acid in a distillation unit 219 and circulated back to hydrolysis unit 211. Concentrated acid 223 is collected or concentrated further in a further acid distillation unit 224 to produce pure acid 225, such as 99% formic acid. Remaining acidic water 226 is recirculated back to distillation unit 219.

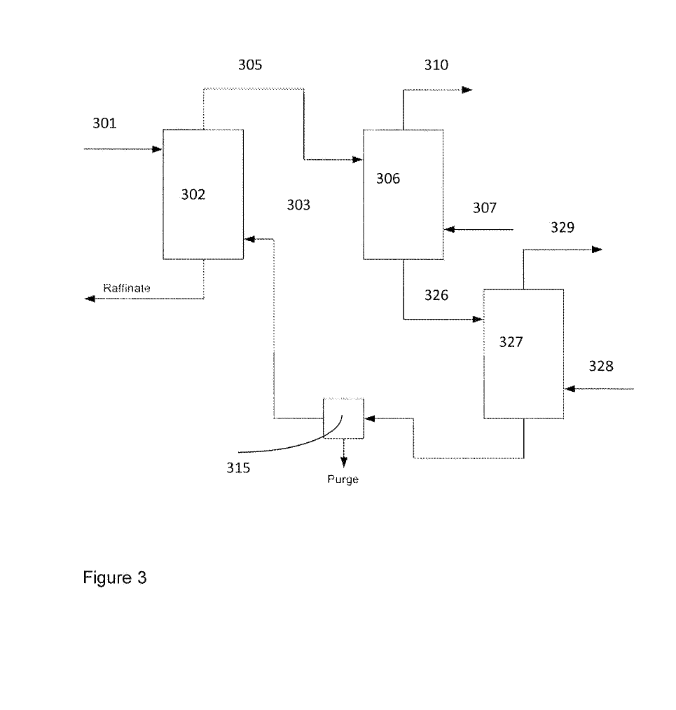

In a preferred embodiment of FIG. 3 a mixture of concentrated organic acids, such as formic acid and levulinic acid, is produced from a dilute aqueous solution thereof. The dilute aqueous acid mixture solution 301 is fed counter currently into an extraction unit 302 together with extractant 303. The organic extract phase 305 containing the formed complexes of the acids and extractant is directed into a first esterification unit 306 together with the first alcohol 307 such as methanol. The formed ester 310, such as methyl formate, is removed from the esterification unit after distillation and directed into further processing according to FIG. 1. The remaining extractant phase 326 is directed to the second esterification unit 327 together with the second alcohol 328 such as ethanol. The formed ester 329 such as ethyl levulinate is removed from the esterification unit 327 after distillation and directed into further processing according to FIG. 2. The remaining extractant is purified in purification unit 315 and recycled back to extraction unit 302.

The following non-limiting examples are disclosed merely for further illustrating the present invention.

### EXAMPLES

### Example 1

Boiling points of selected water soluble C1-C5 carboxylic acids in the form of free acids and methyl esters are depicted in table 1 for pure compounds. The boiling point interval within the mixture in the form of free acids under atmospheric pressure is 84° C., and respectively in the form of methyl esters 95° C. indicating larger separation available for the latter set. The acid recovery in the form of esters is achieved both at lower temperature and with a better separation compared to recovery in the form of free acids even if no complexing with the extractant takes place.

This is an evidence that it is clearly energetically more favourable to recove the acids as esters compared to recovery in acid form.

TABLE 1

| | Boiling point | Boiling point |
|---|---|---|
| | at 1 atm as | at 1 atm as |
| Acid | free acid | methyl ester |
| formic acid | 101 | 33 |
| acetic acid | 117 | 58 |
| propionic acid | 141 | 79 |
| butyric acid | 162 | 103 |
| valeric acid | 185 | 128 |

### Examples 2-5

Extraction

A Scheibel column was filled with an aqueous solution containing 3.5 wt-% formic acid (Kemira) from the top of the column at the rate of 3.93 kg/h. Cyanex 932 (Cytec) solution was fed to the bottom of the column at the rate of 0.998 kg/h. Agitation speed was 350 rpm and the temperature of the column was in the range of 25-28° C. Extraction solution was separated and taken out of the column at the rate of 1.08 kg/h. It contained 9.9 wt-% formic acid (calculated as pure) and 3.4 wt-% water in Cyanex 923. The recovery yield of formic acid in Cyanex 923 was 78%.

Esterification

500.23 g of the obtained extraction solution was mixed with 105.73 g of methanol in a glass reactor heated with circulating silicone oil. The reactor was equipped with a fractionating distillation column containing structured packing and a cooler, cooled with isopropyl alcohol. The solution was warmed up to 70° C. with continuous mixing under ambient pressure. Distillates comprising methyl formate in methanol were collected and more methanol was stepwise introduced into the reactor below the liquid surface level.

The consumption of methanol was recorded and methyl formate formation was quantified from distillates and from the distillation bottom with GC.

Table 2 shows the uniform quality of the distillate obtained by even introduction of methanol into esterification.

TABLE 2

| | Bottom | | | | | |
|---|---|---|---|---|---|---|
| | tem- | Top tem- | | Distillate | Methyl | Methyl |
| | perature | perature | Methanol | collected | formate | formate |
| Step | (° C.) | (° C.) | addition | (g) | (wt-%) | (g) |
| I | 20 → 70 | 20 → 62 | 77.4 g | 26.0 | 37.2 | 9.7 |
| | | | (30 g/h) | | | |
| II | 70 | 62 → 63 | 38.8 g | 18.7 | 33.6 | 6.3 |
| | | | (55 g/h) | | | |
| III | 70 | 63 | 36.5 g | 19.4 | 28.2 | 5.5 |
| | | | (60 g/h) | | | |
| IV | 70 | 63 → 64 | 27.4 g | 24.3 | 22.2 | 5.4 |
| | | | (40 g/h) | | | |

The experiment was carried on only for three hours and table 3 shows the final outcome.

TABLE 3

| | g | mol |
|---|---|---|
| Formic acid in the solution | 49.67 | 1.08 |
| at start | | |
| Total methanol | 285.8 | 8.92 |
| Alcohol/acid | | 8.3 |
| (mol/mol) | | |
| Methyl formate collected | 26.81 | 0.446 |
| Recovery yield (%) | | 41 |

Hydrolysis

Subsequently, the recovered methyl formate was hydrolysed as described in EP0005998 in example from column 4, line 36 to column 6 line 25 into formic acid and methanol which was circulated back to esterification. The concentration of the obtained formic acid was 85% by weight and the overall yield about 40%.

Substitution of the final overpressure distillation in EP0005998 with an underpressure distillation yields even higher final concentration for the recovered formic acid, such as 99%.

### Example 3

Extraction

Extraction was carried out similarly to example 2.

Extraction solution taken out of the column contained 9.0 wt-% formic acid (calculated as pure) and 3.1 wt-% water in Cyanex 923.

Esterification

500.00 g of the obtained extraction solution was mixed with 96.0 g of methanol similarly to example 2. The solution was warmed up to 90° C. with continuous mixing under ambient pressure. Distillates comprising methyl formate in methanol were collected and more methanol was stepwise introduced into the reactor below the liquid surface level. The solution was kept at about 90° C. and the distillation was continued as long as distillate was obtained.

The consumption of methanol was recorded and methyl formate formation was quantified from distillates and from the distillation bottom with GC. Table 4 shows the uniform quality of the distillate and table 5 shows the final outcome.

TABLE 4

| Bottom | | Distillate | Methyl | |
|---|---|---|---|---|
| temperature | Top temperature | collected | formate | Methyl formate |
| (° C.) | (° C.) | (g) | (wt-%) | (g) |
| 20 → 90 → 80 | 20 → 65 → 38 | 250.4 | 25.5 | 63.8 |

TABLE 5

| | g | mol |
|---|---|---|
| Formic acid in the solution | 49.6 | 1.08 |
| at start | | |
| Total methanol | 284.2 | 8.87 |
| Alcohol/acid | | 8.2 |
| (mol/mol) | | |
| Methyl formate collected | 63.8 | 1.06 |
| Recovery yield (%) | | 98 |

The recovery yield is virtually about 100% as there always remains some small residue inside the column and the cooler. This example shows that it is possible to recover nearly all acid from the dilute solution when the extractant solution is heated into efficient temperature.

### Comparative Example 1

Extraction was carried out similarly to example 2.

Extraction solution taken out of the column contained 9.9 wt-% formic acid (calculated as pure) and 3.4 wt-% water in Cyanex 923.

504.33 g of extraction solution was mixed in a class reactor similarly to example 2 but without adding methanol and heated with circulating silicone oil. The reactor was equipped with a fractionating distillation column containing structured packing and a cooler. The solution was kept at 73-87° C. with continuous mixing at 300 mbar. Water was pumped in below the liquid surface level and distillates i.e. formic acid in water were collected.

Formic acid was quantified from the distillates and the distillation bottom with HPLC.

Total amount of distillates was 296.91 g which contained 2.84 g formic acid i.e. recovery yield was 5.7%.

Formic acid forms an azeotrope which has a low boiling point (Ullmann: formic acid (70.5%)-water (29.5%) azeotrope bp. 72 ° C./267 mbar). When azeotrope is not formed formic acid has a boiling point of 105° C. The mixture is easily distilled but the distillate obtained contains about 99% water and only about 1% of formic acid.

### Example 4

An extractant solution comprising 24.83 g 99% formic acid (Kemira) with 103.12 g 99% N,N-dibutylformamide (Alfa Aesar) i.e. 19% by weight of formic acid was introduced into a round bottomed flask which was equipped with a magnetic stirrer, temperature probes, a Vigreaux column and a distillate condenser cooled with water. Subsequently, 34.02 g methanol (J. T. Baker, 99+%) was added into this flask. The solution temperature was increased gradually to 90° C. and the distillates were collected, weighed and analyzed for methyl formate (GC).

Table 6 shows the quality of the distillate obtained by batch type introduction of methanol into esterification.

TABLE 6

| | Bottom | | Distillate | Methyl | Methyl |
|---|---|---|---|---|---|
| | temperature | Top temperature | collected | formate | formate |
| Step | (° C.) | (° C.) | (g) | (wt-%) | (g) |
| I | 20 → 69 | 20 → 24 | 0 | 0 | 0 |
| II | 69-72 | 33-36 | 7.64 | 93.0 | 7.10 |
| III | 72 → 90 | 41 → 64 | 25.63 | 4.8 | 1.23 |

Mass loss during distillation was 3.93 g which originates probably mainly from escaped methyl formate. Table 7 shows the final outcome.

TABLE 7

| | g | mol |
|---|---|---|
| Formic acid in the solution | 24.58 | 0.534 |
| at start | | |
| Methanol | 34.02 | 1.06 |
| Alcohol/acid | | 2.0 |
| (mol/mol) | | |
| Methyl formate collected | 8.33 | 0.139 |
| Recovery yield (%) | | 26 |

In this small scale batch type reaction part of the methanol was distilled together with the methyl formate. Using a non-optimized methanol to formic acid ratio of 2 a quarter of the formic acid could be recovered.

### Comparative Example 2

Similarly to example 4, an extractant solution comprising 21.93 g 99% formic acid (Kemira) with 98.58 g 99% N,N-dibutylformamide (Alfa Aesar) i.e. 18% by weight of formic acid was introduced into a round bottomed flask which was equipped with a magnetic stirrer, temperature probes, a Vigreaux column and a distillate condenser cooled with water. This time, no methanol was added. Temperature was first increased gradually to 153° C. at 1 bar which temperature is approaching the decomposition temperature of formic acid. Subsequently, the solution was let cool and it was warmed up the second time to 103° C. at about 5 mbar which is close to the boiling point of the solvent, 120° C. at 20 mbar.

No distillate was obtained. Mass loss during the experiment was 0.89 g.

### Example 5

An extractant solution comprising 29.65 g 100% acetic acid (AnalR Normapur) and 101.74 g 99% N,N-dibutylformamide (Alfa Aesar) i.e. 23% by weight of acetic acid was introduced into a round bottomed flask similarly to example 4. Subsequently, 32.30 g methanol (J. T. Baker, 99+%) was added into this flask. The solution temperature was increased gradually to 90° C. and the distillates were collected, weighed and analyzed for methyl acetate (GC).

Table 8 shows the quality of the distillate obtained by the batch type introduction of methanol into esterification.

TABLE 8

| | Bottom | | Distillate | Methyl | Methyl |
|---|---|---|---|---|---|
| | temperature | Top temperature | collected | acetate | acetate |
| Step | (° C.) | (° C.) | (g) | (wt-%) | (g) |
| I | 20 → 86 | 20 → 62 | 10.43 | 21.1 | 2.20 |
| II | 86 → 95 | 62 → 65 | 13.13 | 10.3 | 1.35 |

Mass loss during distillation was 2.56 g due to probably loss of both methyl acetate and methanol. Table 9 shows the final outcome.

TABLE 9

| | g | mol |
|---|---|---|
| Acetic acid in the solution at start | 29.65 | 0.494 |
| Methanol | 32.30 | 1.01 |
| Alcohol/acid | | 2.0 |
| (mol/mol) | | |
| Methyl acetate collected | 3.55 | 0.048 |
| Recovery yield (%) | | 10 |

### Comparative Example 3

Similarly to example 5, an extractant solution comprising 30.80 g 99% acetic acid (AnalR Normapur) and 102.42 g 99% N,N-dibutylformamide (Alfa Aesar) i.e. 23% by weight of acetic acid was introduced into a round bottomed flask similarly to example 4. This time, no methanol was added. Temperature was increased gradually to 160° C. at 1 bar. No distillate was obtained even though the boiling point of pure acetic acid is 117° C. The solution was let to cool and it was subsequently warmed up again to 104° C. at about 5 mbar. This vacuum distillation produced 3.97 g distillate. It contained impure acid, wherefrom 84.6% was acetic acid and the rest was mainly solvent. Recovery yield of the acid was 11%.

Mass loss during the experiment was 1.56 g.

1. A method for recovery of at least one organic acid from a dilute aqueous solution thereof characterized in that a complex between said organic acid and an extractant is formed by contacting said solution with a reactive extractant, and dissolving the formed complex into said extractant thus forming an extractant phase, and removing said organic acid from the formed complex by esterification using an alcohol.

2. A method according the claim 1 characterized in that said organic acid comprises a carboxylic acid or mixtures of at least two carboxylic acids.

3. A method according to claim 1 or 2 characterized in that said organic acid is selected from the group of formic acid, acetic acid, propionic acid, levulinic acid and mixtures thereof.

4. A method according to any one of the claims 1-3 characterized in that the total organic acid concentration of said dilute aqueous solution is less than 40% by weight, preferably less than 15%, more prefereably from 0.01 to 10%, most preferably from 0.5 to 5%.

5. A method according to any one of the claims 1-4 characterized in that said dilute aqueous solution originates from biomass.

6. A method according to any one of the claims 1-5 characterized in that said reactive extractant is selected from the group of trialkyl phosphine oxides, N,N-dialkyl amides, trialkyl amines and ditrialkylformamides.

7. A method according to claims 1-6 characterized in that said extractant is counter currently contacted with an alcohol using an ion exchange resin column wherein said esterification takes place.

8. A method according to any one of the claims 1-7 characterized in that said esterification takes place under elevated temperature and ambient pressure.

9. A method according to any one of the claims 1-8 characterized in that said alcohol is C1-C6 alcohol, preferably methanol or ethanol.

10. A method according to any one of the claims 1-9 characterized in that a catalyst is used in said esterification selected from the group of p-toluenesulphonic acid, mineral acid and acidic ion exchange resin.

11. A method according to any one of the claims 1-10 characterized in that said esterification reaction is an equilibrium reaction wherefrom formed ester(s) is(are) continuously removed.

12. A method according to any one of the claims 1-11 characterized in that said formed ester(s) is(are) separated from extractant solution by fractionating distillation.

13. A method according to any one of the claims 1-12 characterized in that said formed and separated ester(s) is(are) further hydrolysed into corresponding acid(s) and alcohol.

14. A method according to any one of the claims 1-13 characterized in that said alcohol is recycled into said esterification.

15. A method according to any one of the claims 1-14 characterized in that extractant wherefrom ester(s) has(have) been removed is recycled to extraction.

16. An arrangement for use in the method of claim 1 characterized in that it comprises
an extraction unit for carrying out an extraction of at least one organic acid from dilute aqueous solution thereof with an extractant forming a complex between said organic acid and said extractant, connected to
at least one esterification unit for carrying out esterification of said extracted acid from said complex, connected optionally to
a hydrolysis unit for carrying out hydrolysis of said esterified organic acid into free organic acid and alcohol.
